(19) United States
(12) Patent Application Publication
(10) Pub. No.: US 2005/0281757 A1
(43) Pub. Date: Dec. 22, 2005

(54) Oral care film

(76) Inventors: Sayed Ibrahim, Somerset, NJ (US); Suman K. Chopra, Dayton, NJ (US); Michael Prencipe, West Windsor, NJ (US)

Correspondence Address: HARNESS, DICKEY, & PIERCE, P.L.C, 7700 BONHOMME, STE 400, ST. LOUIS, MO, 63105, US

(21) Appl. No.: 10/870,848

(22) Filed: Jun. 17, 2004

(51) Int. Cl. A61K 7/16; A61K 9/00

(52) U.S. Cl. 424/49

(57) ABSTRACT

A composition for delivery of an oral care substance to a dental surface upon application of the composition thereto is a flexible film comprising the oral care substance dispersed in a film-forming effective amount of a polymeric matrix having a hydrophilic component, e.g., vinylpyrrolidone (VP), and a hydrophobic component, e.g., vinyl acetate (VA), in a weight ratio selected such that the film is substantially dissolvable in saliva in a period of time effective for delivery of the oral care substance. The polymeric matrix illustratively comprises a poly(VP/VA) copolymer having a VP/VA weight ratio of about 90:10 to about 10:90.

### FIELD

This invention relates to a composition useful for oral care, more particularly care of a dental surface.

### BACKGROUND

It is known to deliver an oral care substance, for example a tooth whitening substance, to a dental surface by means of a removable strip of flexible material having coated thereon a composition such as a gel containing the oral care substance. See for example U.S. Pat. No. 5,891,453 and No. 6,096,328, both to Sagel et al.

Another means of delivery of an oral care substance to a dental surface is in a form of a liquid or gel that can be applied to the surface, for example by painting with a soft applicator. For example, on the packaging of Colgate® Simply White® Night clear whitening gel sold by Colgate-Palmolive Co., New York, N.Y., the user is directed to apply a thin layer of the gel to a dental surface. The gel typically remains in place until removed, for example by toothbrushing.

U.S. Patent Application Publication No. 2003/0124065 discloses compositions said to be useful for dental stain removal and prevention, comprising a vinylpyrrolidone/vinyl acetate copolymer (PVP/VA) having a vinylpyrrolidone/vinyl acetate weight ratio of 30:70 to 90:10, for example 60:40.

Patents and publications cited above are incorporated herein by reference.

It would be of benefit in the art to have a delivery system for oral care substances in a form of a composition that would remain on a dental surface for a long enough period to be efficacious yet would not have to be mechanically removed at the end of such period.

### SUMMARY

Now provided is a composition for delivery of an oral care substance to a dental surface upon application of the composition thereto, the composition being a flexible film comprising the oral care substance dispersed in a film-forming effective amount of a polymeric matrix having a hydrophilic component and a hydrophobic component in a weight ratio selected such that the film is substantially dissolvable in saliva in a period of time effective for delivery of the oral care substance.

In one embodiment the polymeric matrix comprises polymerized vinylpyrrolidone (VP) and vinyl acetate (VA) monomers in a VP/VA weight ratio of about 90:10 to about 10:90. The VP and VA monomers can be present in a physical mixture of separate homopolymers, i.e., polyvinylpyrrolidone (PVP) and polyvinyl acetate (PVA) respectively, or they can be present together in a PVP/VA copolymer. In either case, adjusting the weight ratio of the more hydrophilic VP to the more hydrophobic VA monomers enables dissolution time to be controlled for optimum delivery of the oral care substance.

There is also provided a method of delivering an oral care substance to a dental surface, the method comprising placement of a composition as described above on the surface with sufficient pressure to promote adhesion of the composition to the surface.

### DETAILED DESCRIPTION

A “dental surface” herein is a surface of a natural tooth or a hard surface of artificial dentition including a crown, cap, filling, bridge, denture, dental implant and the like.

An “orally acceptable” compound, composition or vehicle is one that is not harmful to a mammal in amounts disclosed herein when retained in the mouth, without swallowing, for a period sufficient to permit application to a dental surface as required herein. In general, such a compound, composition or vehicle is not harmful even if unintentionally swallowed.

Classification herein of an ingredient as an active or a carrier ingredient is made for clarity and convenience, and no inference should be drawn that a particular ingredient necessarily functions in the composition in accordance with its classification herein. Furthermore, a particular ingredient can serve a plurality of functions, thus disclosure of an ingredient herein as exemplifying one functional class does not exclude the possibility that it can also exemplify another functional class.

Among useful oral care substances or actives are those addressing, without limitation, appearance and structural changes to teeth, treatment and prevention of plaque, calculus, dental caries, cavities, abscesses, inflamed and/or bleeding gums, gingivitis, oral infective and/or inflammatory conditions in general, tooth sensitivity, halitosis and the like. Thus, a composition of the invention can contain one or more actives such as whitening agents, fluoride ion sources, antimicrobial agents, desensitizing agents, anticalculus (tartar control) agents, stannous ion sources, zinc ion sources, antioxidants, sialagogues, breath-freshening agents, antiplaque agents, anti-inflammatory agents, periodontal agents, analgesics and nutrients.

Actives useful herein are normally present in the composition in amounts selected to be safe and effective. A “safe and effective” amount in the present context is an amount sufficient to provide a desired benefit, for example a therapeutic, prophylactic or cosmetic effect, when the composition is used repeatedly as described herein, without undue side effects such as toxicity, irritation or allergic reaction, commensurate with a reasonable benefit/risk ratio. Such a safe and effective amount will usually, but not necessarily, fall within ranges approved by appropriate regulatory agencies. A safe and effective amount in a specific case depends on many factors, including the particular benefit desired or condition being treated or sought to be prevented, the particular subject using, or being administered, the composition, the frequency and duration of use, etc. Oral care substances are typically present in a total amount of about 0.01% to about 80%, for example about 0.05% to about 60%, about 0.1% to about 50%, or about 0.5% to about 40%, by weight of the composition.

In one embodiment the composition comprises as an oral care substance an orally acceptable whitening agent. One or more such agents can be present. Suitable whitening agents include peroxy compounds, chlorine dioxide, chlorites and hypochlorites (e.g., chlorites and hypochlorites of alkali and alkaline earth metals such as lithium, potassium, sodium, magnesium, calcium and barium).

Suitable peroxy compounds include hydrogen peroxide, peroxides of alkali and alkaline earth metals, organic peroxy compounds and peroxy acids and salts thereof. Any orally acceptable compound that delivers a perhydroxy (OOH⁻) ion is useful. A peroxy compound can optionally be present in a form of a polymer-peroxide complex, for example a polyvinylpyrrolidone-hydrogen peroxide (PVP-H₂O₂) complex, or encapsulated in a polymer microsphere or nanosphere, for example hydrogen peroxide encapsulated in an allyl methacrylate cross-linked polymer (e.g., Poly-Pore™ of Amcol Health & Beauty Solutions).

Peroxides of alkali and alkaline earth metals include lithium peroxide, potassium peroxide, sodium peroxide, magnesium peroxide, calcium peroxide and barium peroxide.

Organic peroxy compounds include, for example, carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, monoperoxyphthalate and the like.

Peroxy acids and their salts include organic peroxy acids such as alkyl peroxy acids and monoperoxyphthalate, as well as inorganic peroxy acid salts including persulfate, dipersulfate, percarbonate, perphosphate, perborate and persilicate salts of alkali and alkaline earth metals such as lithium, potassium, sodium, magnesium, calcium and barium. Another useful peroxy compound is sodium pyrophosphate peroxyhydrate.

According to the present embodiment, at least one whitening agent is present in the composition in a total amount effective to result in whitening of a dental surface when applied in accordance with the disclosure herein. Peroxy compounds can illustratively be present in a total hydrogen peroxide equivalent amount of about 0.1% to about 50%, for example about 1% to about 40% or about 5% to about 25%, by weight of the composition. Illustratively, a PVP-H₂O₂ complex can be present in an amount of about 5% to about 80% by weight of the composition. Illustratively, sodium percarbonate can be present in an amount of about 0.1% to about 75% by weight of the composition.

In another embodiment the composition comprises as an oral care substance an orally acceptable source of fluoride ions. One or more such sources can be present. Suitable sources of fluoride ions include fluoride, monofluorophosphate and fluorosilicate salts. Any such salt that is orally acceptable can be used, including without limitation alkali metal (e.g., potassium, sodium), ammonium, stannous and indium salts and the like. Water-soluble fluoride-releasing salts are typically used. One or more fluoride-releasing salts are optionally present in an amount providing a total of about 100 to about 20,000 ppm, about 200 to about 5,000 ppm, or about 500 to about 2,500 ppm, fluoride ions. Where sodium fluoride is the sole fluoride-releasing salt present, illustratively an amount of about 0.01% to about 5%, about 0.05% to about 1% or about 0.1% to about 0.5%, sodium fluoride by weight can be present in the composition.

In another embodiment the composition comprises as an oral care substance an orally acceptable antimicrobial (e.g., antibacterial) agent. One or more such agents can be present. Suitable antimicrobial agents include without limitation triclosan (5-chloro-2-(2,4-dichlorophenoxy)phenol), 2,2′-dihydroxy-5,5′-dibromodiphenyl ether, 8-hydroxyquinoline and salts thereof, copper (II) compounds such as copper (II) chloride, fluoride, sulfate and hydroxide, zinc ion sources such as zinc citrate, zinc sulfate, zinc glycinate and sodium zinc citrate, phthalic acid and salts thereof such as magnesium monopotassium phthalate, hexetidine, octenidine, sanguinarine, benzalkonium chloride, salicylanilide, domiphen bromide, alkylpyridinium chlorides such as cetylpyridinium chloride (CPC) (including combinations of CPC with zinc and/or enzymes), tetradecylpyridinium chloride and N-tetradecyl-4-ethylpyridinium chloride, iodine, sulfonamides, bisbiguanides such as alexidine, chlorhexidine and chlorhexidine digluconate, phenolics, piperidino derivatives such as delmopinol and octapinol, magnolia extract, grapeseed extract, phenol, thymol, eugenol, menthol, geraniol, carvacrol, citral, eucalyptol, catechol, 4-allylcatechol, hexyl resorcinol, halogenated bisphenolics such as 2,2′-methylene bis(4-chloro-6-bromophenol), methyl salicylate, antibiotics such as augmentin, amoxicillin, tetracycline, doxycycline, minocycline, metronidazole, neomycin, kanamycin and clindamycin, and the like. A further illustrative list of useful antibacterial agents is provided in U.S. Pat. No. 5,776,435 to Gaffar et al., incorporated herein by reference. One or more antimicrobial agents are optionally present in an antimicrobial effective total amount, typically about 0.05% to about 10%, for example about 0.1% to about 3% by weight, of the composition.

In another embodiment the composition comprises as an oral care substance an orally acceptable desensitizing, or tooth sensitivity protecting, agent. One or more such agents can be present. Suitable desensitizing agents include without limitation potassium salts such as potassium citrate, potassium tartrate, potassium chloride, potassium sulfate and potassium nitrate. Another suitable desensitizing agent is sodium nitrate. Alternatively or in addition a local or systemic analgesic such as aspirin, codeine, acetaminophen, sodium salicylate or triethanolamine salicylate can be used. One or more densitizing agents and/or analgesics are optionally present in a desensitizing and/or analgesic effective amount, typically about 0.05% to about 5%, for example about 0.1% to about 3% by weight, of the composition.

In another embodiment the composition comprises as an oral care substance an orally acceptable anticalculus agent. One or more such agents can be present. Suitable anticalculus agents include without limitation phosphates and polyphosphates (for example pyrophosphates), polyaminopropanesulfonic acid (AMPS), zinc citrate trihydrate, polypeptides such as polyaspartic and polyglutamic acids, polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts. Useful inorganic phosphate and polyphosphate salts illustratively include monobasic, dibasic and tribasic sodium phosphates, sodium tripolyphosphate, tetrapolyphosphate, mono-, di-, tri- and tetrasodium pyrophosphates, disodium dihydrogen pyrophosphate, sodium trimetaphosphate, sodium hexametaphosphate and the like, wherein sodium can optionally be replaced by potassium or ammonium. Other useful anticalculus agents include polycarboxylate polymers and polyvinyl methyl ether/maleic anhydride (PVME/MA) copolymers, such as those available under the Gantrez™ brand from ISP, Wayne, N.J. One or more anticalculus agents are optionally present in the composition in an anticalculus effective total amount, typically about 0.01% to about 50%, for example about 0.05% to about 25% or about 0.1% to about 15% by weight.

In another embodiment the composition comprises as an oral care substance an orally acceptable stannous ion source useful, for example, in helping reduce gingivitis, plaque, calculus, caries or sensitivity. One or more such sources can be present. Suitable stannous ion sources include without limitation stannous fluoride, other stannous halides such as stannous chloride dihydrate, stannous pyrophosphate, organic stannous carboxylate salts such as stannous formate, acetate, gluconate, lactate, tartrate, oxalate, malonate and citrate, stannous ethylene glyoxide and the like. One or more stannous ion sources are optionally and illustratively present in a total amount of about 0.01% to about 10%, for example about 0.1% to about 7% or about 1% to about 5% by weight of the composition.

In another embodiment the composition comprises as an oral care substance an orally acceptable zinc ion source useful, for example, as an antimicrobial, anticalculus or breath-freshening agent. One or more such sources can be present. Suitable zinc ion sources include without limitation zinc citrate, zinc sulfate, zinc glycinate, sodium zinc citrate and the like. One or more zinc ion sources are optionally and illustratively present in a total amount of about 0.05% to about 3%, for example about 0.1% to about 1%, by weight of the composition.

In another embodiment the composition comprises as an oral care substance an orally acceptable antioxidant. One or more antioxidants can be present in an antioxidant effective total amount. Suitable antioxidants include without limitation butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin and the like.

In another embodiment the composition comprises as an oral care substance an orally acceptable sialagogue (saliva stimulating agent) useful for example in amelioration of dry mouth. One or more such agents can be present in a saliva stimulating effective total amount. Suitable sialagogues include without limitation food acids such as citric, lactic, malic, succinic, ascorbic, adipic, fumaric and tartaric acids.

In another embodiment the composition comprises as an oral care substance an orally acceptable breath-freshening agent. One or more such agents can be present in a breath-freshening effective total amount. Suitable breath-freshening agents include without limitation zinc salts such as zinc gluconate, zinc citrate and zinc chlorite, a-ionone and the like.

In another embodiment the composition comprises as an oral care substance an orally acceptable antiplaque, including plaque disrupting, agent. One or more such agents can be present in an antiplaque effective total amount. Suitable antiplaque agents include without limitation stannous, copper, magnesium and strontium salts, dimethicone copolyols such as cetyl dimethicone copolyol, papain, glucoamylase, glucose oxidase, urea, calcium lactate, calcium glycerophosphate, strontium polyacrylates and chelating agents such as citric and tartaric acids and alkali metal salts thereof.

In another embodiment the composition comprises as an oral care substance an orally acceptable anti-inflammatory agent. One or more such agents can be present in an anti-inflammatory effective total amount. Suitable anti-inflammatory agents include without limitation steroidal agents such as flucinolone and hydrocortisone, and nonsteroidal agents (NSAIDs) such as ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone and phenylbutazone. One or more anti-inflammatory agents are optionally present in the composition in an anti-inflammatory effective amount.

In another embodiment the composition comprises as an oral care substance an orally acceptable nutrient. One or more nutrients can be present. Suitable nutrients include vitamins, minerals and amino acids.

The polymeric matrix wherein the oral care substance is dispersed is selected to provide a film that is substantially dissolvable in saliva in a period of time effective for delivery of the oral care substance, when the film is placed in contact with a dental surface. By increasing the proportion of hydrophilic monomers in the polymer matrix, dissolution time of the film can be shortened. Conversely, by increasing the proportion of hydrophobic monomers in the polymer matrix, dissolution time of the film can be lengthened. One of ordinary skill will be able, by routine experimentation based on the disclosure herein, to select suitable hydrophilic and hydrophobic monomers at a suitable weight ratio to provide a dissolution time appropriate for delivery of a particular oral care substance.

Desirably, the dissolution time in saliva is about 5 to about 60 minutes, for example about 5 to about 30 minutes, about 10 to about 30 minutes or about 15 to about 25 minutes. Longer or shorter dissolution times can be useful in specific circumstances.

To provide such dissolution times, the weight ratio of hydrophilic to hydrophobic monomers is typically about 90:10 to about 10:90, for example about 75:25 to about 20:80, or about 60:40 to about 30:70.

In one embodiment the hydrophilic monomers are selected from the group consisting of vinylpyrrolidone, vinyl alcohol, acrylic acid, methacrylic acid and ethylene oxide; and the hydrophobic monomers are selected from the group consisting of methyl methacrylate, ethyl acrylate, dimethylaminoethyl methacrylate, propylene oxide, vinyl caprolactam, vinyl acetate, methacrylamide and vinylimadazole.

The invention is illustrated herein by particular reference to vinylpyrrolidone (VP) as a hydrophilic monomer and vinyl acetate (VA) as a hydrophobic monomer.

In one embodiment the polymeric matrix comprises a physical mixture of polyvinylpyrrolidone (PVP) and polyvinyl acetate (PVA) in a PVP/PVA weight ratio of about 90:10 to about 10:90, for example about 75:25 to about 20:80, or about 60:40 to about 30:70. Such a physical mixture can readily be prepared from PVP and PVA products, or can be obtained as a premixed product. For example, Kollicoat® SR 30 D of BASF contains PVP and PVA in a 10:90 approximate weight ratio; the ratio can be adjusted upwards by addition of an appropriate amount of PVP if desired.

In another embodiment the polymeric matrix comprises a copolymer of vinylpyrrolidone (VP) and vinyl acetate (VA) monomers in a VP/VA weight ratio of about 90:10 to about 10:90, for example about 75:25 to about 20:80, or about 60:40 to about 30:70. Such a copolymer is referred to herein as a poly(vinylpyrrolidone/vinyl acetate) or PVP/VA copolymer. Both random and block copolymers of VP and VA can be useful.

Suitable PVP/VA copolymers typically have an average molecular weight of about 10,000 to about 1,000,000, for example about 20,000 to about 200,000 or about 27,000 to about 70,000. Illustrative PVP/VA products that can be used include Kollidon® VA 64 of BASF, having a VP/VA weight ratio of 60:40, and Luviskol® VA 37E of BASF, having a VP/VA weight ratio of 30:70. PVP/VA products can be supplied as a powder or in a solvent such as ethanol, for example at a concentration of about 30% to about 60%. More than one PVP/VA copolymer can be present in the polymer matrix if desired. For example, to fine-tune the VP/VA weight ratio in a range from 60:40 to 30:70 for optimum dissolution rate and hence optimum delivery of an oral care substance, a mixture of 60:40 and 30:70 copolymers (e.g., Kollidon® VA 64 and Luviskol® VA 37E respectively) can be prepared in any desired proportion.

A PVP/VA copolymer can be present in an amount of about 0.5% to about 70% by weight of the composition. It will generally be found useful to include the PVP/VA copolymer in an amount of at least about 5%, or at least about 10%, by weight. Suitably, the amount of PVP/VA copolymer can be about 20% to about 65% by weight of the composition. Where the PVP/VA copolymer is the sole film-forming material in the composition, relatively large amounts in the ranges given above will generally be found necessary. Where the PVP/VA copolymer is accompanied by other film-forming polymers, lesser amounts can be useful.

The polymer matrix as a whole (comprising, for example, a PVP/VA copolymer and optionally additional polymeric materials) is present in a film-forming amount, generally at least about 20% and up to about 95% by weight of the composition. In various embodiments the polymer matrix constitutes about 25% to about 90%, about 30% to about 80%, or about 35% to about 65%, by weight of the composition.

Optional additional polymers in the composition, as a component of or separate from the polymeric matrix wherein the oral care substance is dispersed, can affect such properties of the composition as dissolution rate, adhesiveness to the dental surface, flexibility, mechanical strength, compatibility with the oral care substance, etc., and can include without limitation PVP, polyethylene oxide, methylcellulose, ethylcellulose, carbomers (carboxyvinyl polymers), polyacrylates etc. The term “polyacrylate” herein encompasses polymers and copolymers having monomeric units selected from acrylic acid, esters and amides and methacrylic acid, esters and amides.

Polyacrylates useful as optional additional polymers are operable to form a film, preferably when cast on a substrate from a solution. In one embodiment, a polyacrylate component comprises a copolymer of one or more first monomeric units selected from the group consisting of acrylic acid, methacrylic acid and combinations thereof, and one or more second monomeric units selected from the group consisting of acrylates, acrylamides and combinations thereof.

Illustratively, the first monomeric units in such a copolymer comprise methacrylic acid or β-methacrylic acid.

Acrylates useful as second monomeric units in such a copolymer include methyl acrylate, ethyl acrylate, n-butyl acrylate, isobutyl acrylate, t-butyl acrylate, 2-ethylhexyl acrylate, lauryl acrylate, tridecyl acrylate, cetyl acrylate, stearyl acrylate, cyclohexyl acrylate, benzyl acrylate, isobornyl acrylate, 2-methoxyethyl acrylate, 2-ethoxyethyl acrylate, 2-phenoxyethyl acrylate, tetrahydrofurfuryl acrylate, 2-hydroxyethyl acrylate, 2-hydroxypropyl acrylate, 4-hydroxybutyl acrylate, dimethylaminoethyl acrylate, glycidyl acrylate, allyl acrylate and 1,4-butanediol acrylate. Diacrylate monomers include the diacrylates of 1,4-butanediol, 1,6-hexanediol, tetraethylene glycol, tripropylene glycol and ethoxylated bisphenol-A. Triacrylate monomers include the triacrylates of trimethylol propane, ethoxylated, glyceryl propoxy, and pentaerythritol.

Methacrylates useful as second monomeric units in such a copolymer include methyl methacrylate, ethyl methacrylate, n-butyl methacrylate, isobutyl methacrylate, t-butyl methacrylate, 2-ethylhexyl methacrylate, lauryl methacrylate, tridecyl methacrylate, cetyl methacrylate, stearyl methacrylate, cyclohexyl methacrylate, benzyl methacrylate, isobornyl methacrylate, 2-methoxyethyl methacrylate, 2-ethoxyethyl methacrylate, 2-phenoxyethyl methacrylate, tetrahydrofurfuryl methacrylate, 2-hydroxyethyl methacrylate, 2-hydroxypropyl methacrylate, dimethylaminoethyl methacrylate, glycidyl methacrylate, allyl methacrylate, ethylene glycol methacrylate, triethylene glycol methacrylate, tetraethylene glycol methacrylate, 1,3-butyleneglycol methacrylate, 1,6-hexanediol methacrylate, trimethylolpropane methacrylate, ethoxyethyl methacrylate and trifluoroethyl methacrylate.

Acrylamides and methacrylamides useful as second monomeric units in such a copolymer include mono- and di(C₁₋₃₀)-alkyl acrylamides and methacrylamides such as those of methyl, ethyl, propyl, butyl, pentyl, hexyl and the like. N-substituted acrylamides can also include N-ethylacrylamide, N-t-butylacrylamide, N-t-octylacrylamide, N-octylacrylamide, N-decylacrylamide, N-dodecylacrylamide and the corresponding N-substituted methacrylamides. Other N-substituted acryl amides include N-hydroxymethylacrylamide, N-isopropylacrylamide, N-methylacrylamide, N,N′-methylenebisacrylamide, N-isobutoxymethylacrylamide, N,N-dimethylacrylamide and 2-acrylamido-2-methylpropanesulfonic acid.

Suitable polyacrylates illustratively include copolymers of ethyl acrylate and methyl methacrylate (e.g., Kollicoat® EMM 30 D of BASF, poly(ethyl acrylate, methyl methacrylate), 2:1) and copolymers of methacrylic acid and ethyl acrylate (e.g., Kollicoat® MAE 30 DP of BASF, poly(methacrylic acid, ethyl acrylate), 1:1).

Other polyacrylates useful herein include poly(2-hydroxyethyl methacrylate), otherwise known as poly-HEMA, available for example from Polysciences, Inc.; water-soluble acrylic acid copolymers such as MG-0560, MG-0580, and MG-0607 of Dow Corning; and water-swellable polyacrylate polymers such as those of the Eudragit™ series (including E, L, S, RL, RS and NE) of Rohm Pharma, for example Eudragit™ E100.

Polyacrylate copolymers can additionally comprise one or more third monomeric units selected from the group consisting of siloxanes, vinyl esters of C₅₋₁₂ alcohols, and combinations thereof. A siloxane monomer unit includes a silicon-oxygen-silicon bond referred to as a siloxane bond and can be substituted with hydrocarbon radicals attached directly via a carbon atom thereof to the silicon atoms. The most common hydrocarbon radicals are alkyl radicals, especially C₁₋₁₀ alkyl radicals. The resulting silicones can be polymerized or polycondensed. Compounds incorporating the siloxane bond include, but are not limited to, hexamethyldisiloxane, octamethyltrisiloxane, linear siloxanes such as dimethicone and aromatic siloxanes. Siloxane-containing polymers among those useful herein include poly(dimethyl siloxane)-g-polyacrylate polymers, such as Silicone “Plus” Polymer VS80™ of 3M Corporation. Vinyl esters suitable as third monomeric units include vinyl pentanoate, vinyl hexanoate, vinyl heptanoate, vinyl octanoate, vinyl nonanoate, vinyl decanoate and vinyl dodecanoate.

Other optional polymer components comprise salts and esters of acetic acid. A non-limiting example of such a salt is zinc acetate. Esters include those with alkyl, aryl and vinyl groups and the like. In one embodiment a polymer component comprises a terpolymer of vinyl acetate, crotonic acid and vinyl neodecanoate (e.g., Luviset™ CAN of BASF). Other terpolymers include a terpolymer of t-butyl acrylate, methacrylic acid and dimethicone copolyol (e.g., Luviflex™ Silk of BASF), a terpolymer of N-t-butyl acrylamide, ethyl acrylate and acrylic acid (e.g., Ultrahold™ Strong of BASF), and a terpolymer of ethyl acrylate, t-butyl acrylate and methacrylic acid (e.g., Luvimer™ of BASF).

Polymers optionally included to enhance adhesiveness are typically alcohol soluble, substantially water insoluble, and stable to the oral care active (e.g., peroxy compound). Such polymers should be substantive (i.e., capable of firm adherence) to teeth or other surfaces of the oral cavity. Such polymers can comprise a calcium complexation moiety and a hydrophobic moiety. Acrylic acid and methacrylic acid monomers can illustratively function as calcium complexation moieties. It is believed, without being bound by theory, that such polymers provide a saliva-resistant film that is secured against a dental surface, for example by inducing additional interaction with enamel of teeth through complexation with Ca²⁺ components thereof, such as hydroxyapatite. Such complexation is believed to provide increased adhesion of the composition to teeth.

If included, additional polymers can be present in the composition, excluding any non-dissolvable backing layer if present, in the following amounts (all percentages by weight):

PVP: about 0.5% to about 70%;

polyethylene oxide: about 1% to about 90%;

methylcellulose: about 0.1% to about 50%;

ethylcellulose: about 0.1% to about 40%;

carbomer: about 0.01% to about 40%;

polyacrylate: about 0.1% to about 50%.

Addition of water-soluble polymers such as PVP or polyethylene oxide can provide enhanced adhesion to the dental surface but at the same time result in faster dissolution of the film and thus faster delivery of the oral care substance. On the other hand, addition of less soluble polymers such as methylcellulose, ethylcellulose, carbomer or polyacrylate can retard dissolution of the film. Relative amounts of these various polymers can be adjusted to provide an acceptable balance of properties such as dissolution rate, adhesiveness, flexibility and mechanical strength.

Optionally, a low molecular weight polyethylene glycol (PEG), for example a PEG having an average molecular weight of about 300 to about 1000, such as PEG 600, can be included in the composition, for example as a plasticizer. If included, a low molecular weight PEG can be present in the composition in an amount of about 0.2% to about 40%, for example about 5% to about 20%, by weight.

Alternative plasticizers that can optionally be present include propylene glycol, di- and tripropylene glycols, triacetin, petrolatum, vegetable oils, pegylated (e.g., PEG-40) oils, and the like.

Optionally, one or more natural resins such as rosin (colophony), mastic or shellac can be included in the composition, for example as adhesive agents. If included, such resins can be present in the composition in the following amounts (all percentages by weight):

rosin: about 0.2% to about 40%;

mastic: about 0.5% to about 50%;

shellac: about 0.5% to about 60%.

Optionally, a sweetener can be included in the composition. One or more sweeteners can be present. Use of a sweetener can overcome an unpleasant, for example bitter, taste of an oral care active such as sodium percarbonate. Any orally acceptable natural or artificial, nutritive or non-nutritive sweetener can be used, including without limitation dextrose, polydextrose, sucrose, maltose, dextrin, dried invert sugar, lactose, mannose, xylose, ribose, fructose, galactose, corn syrup (including high fructose corn syrup and corn syrup solids), partially hydrolyzed starch, hydrogenated starch hydrolysate, sorbitol, mannitol, xylitol, maltitol, isomalt, sucralose, aspartame, acesulfame, neotame, D-tryptophan, saccharin and salts thereof (e.g., sodium saccharin), thaumatin, dihydrochalcones, dipeptide-based intense sweeteners, cyclamates (e.g., sodium cyclamate) and the like. One or more sweeteners are optionally present in a total amount depending strongly on the particular sweetener(s) selected, but typically about 0.005% to about 10% by weight of the composition.

In a particular embodiment, the composition comprises sodium saccharin in an amount of about 0.1% to about 5% by weight.

Optionally, a flavorant can be included in the composition. One or more flavorants can be present. Use of a flavorant, alone or in conjunction with a sweetener, can overcome an unpleasant, for example bitter, taste of an oral care active such as sodium percarbonate. Any orally acceptable natural or synthetic flavorant can be used, including without limitation vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or warming effects. Such ingredients illustratively include menthol, menthyl acetate, menthyl lactate, camphor, eucalyptus oil, eucalyptol, anethole, eugenol, cassia, oxanone, α-irisone, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like. If included, one or more flavorants can be present in a total amount of about 0.01% to about 5%, for example about 0.1% to about 2.5% by weight of the composition.

Optionally, a colorant can be included in the composition. One or more colorants can be present. Colorants herein include pigments, dyes, lakes and agents imparting a particular luster or reflectivity such as pearling agents. A colorant can serve a number of functions, including for example to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including without limitation talc, mica, magnesium carbonate, calcium carbonate, magnesium silicate, magnesium aluminum silicate, silica, alumina, hydroxyapatite, titanium dioxide, zinc oxide, red, yellow, brown and black iron oxides, ferric ammonium ferrocyanide, manganese violet, ultramarine, titaniated mica, bismuth oxychloride and the like. If included, one or more colorants can be present in a total amount of about 0.001% to about 20%, for example about 0.01% to about 10% or about 0.1% to about 5% by weight of the composition. Colorants such as alumina, hydroxyapatite and titanium dioxide that provide an opaque white appearance to the film can be especially desirable in matching appearance of teeth.

Oral care actives or other ingredients can optionally be present in the film composition in encapsulated form. For example, peroxy compounds, fluoride, antimicrobial, flavor and/or color ingredients can be encapsulated. Encapsulation can enhance stability of such ingredients upon drying of the film, but permit release of the ingredients upon placement in the humid environment of the oral cavity. Further, encapsulation can protect incompatible ingredients from one another. Encapsulation can also provide an additional means of control of release rate and/or time of an oral care active.

As described below, solvent materials such as ethanol, ethyl acetate and water can to a large extent be removed from the composition during a process of manufacture, but some amount of solvent can remain in the finished film. Amount of organic solvent such as ethanol or ethyl acetate can be zero to about 15%, more typically zero to about 5%, by weight of the film composition. Amount of water can be zero to about 20%, more typically zero to about 5%, by weight of the film composition.

The flexible film can be essentially homogeneous or can comprise a plurality of layers of differing composition. Bilayer or multilayer films can be useful, for example, where it is desired to segregate incompatible ingredients (e.g., where the oral care substance is incompatible with an adhesive, where two or more oral care actives are incompatible with each other, or where an oral care active and an activator that is incompatible therewith are present), or where different release rates of one or more oral care substances are desired.

In one embodiment, the flexible film comprises at least two layers, wherein a first layer comprises the oral care active dispersed in a polymeric matrix as described herein, for example a polymeric matrix comprising a PVP/VA copolymer, and a second layer comprises an adhesive. The layers can optionally be differently colored to facilitate correct orientation of the film on a dental surface by the user. Optionally the composition further comprises a backing layer that is non-dissolvable in saliva. Such a backing layer typically comprises one or more water-insoluble polymers such as ethylcellulose, polyethylene or PET (polyethylene terephthalate).

A dissolvable film useful according to the invention can be prepared by any process known in the art for making polymer-based films, such as for example a casting process. In an illustrative process, the materials to be included in the polymeric matrix (for example a PVP/VA copolymer and optionally other polymers) are first added to a suitable amount of a solvent or mixture of solvents in a vessel and stirred until dissolved or homogeneously dispersed. Suitable solvents include water, ethanol and ethyl acetate. The oral care substance(s) and optionally other ingredients such as sweetener are then added with stirring until dissolved or homogeneously dispersed. The resulting liquid is then cast on a heated surface, where the composition forms a film upon partial or complete evaporation of the solvent or solvent mixture.

The dental surface to which the composition is applied according to the method of the invention can be in a human or nonhuman subject, for example a nonhuman mammalian subject such as a companion animal, for example a dog or cat. In one embodiment the dental surface is a surface of one or more natural teeth, but the method is also applicable to a surface of artificial dentition, for example a crown, a cap, a filling, a bridge, a denture or a dental implant.

In one embodiment a method of whitening a dental surface is provided, the method comprising placing on the surface a film composition of the invention that comprises a whitening agent as described above, wherein during the placing sufficient pressure is applied to promote adhesion of the film to the surface.

Degree of whitening of a dental surface can be observed visually, for example with the aid of color comparison charts, gauges or shade guides, e.g., as described by Browning (2003), Journal of Esthetic Restorative Dentistry 15 Supp. 1, S13-S20, incorporated herein by reference.

Alternatively, staining or inhibition thereof can be measured by colorimetry, using any suitable instrument such as a Minolta Chromameter, e.g., model CR-321 (Minolta Corp., Ramsey, N.J.). The instrument can be programmed, for example, to measure Hunter Lab values or L*a*b* values according to the standard established by the International Committee of Illumination (CIE). The L*a*b* system provides a numerical representation of three-dimensional color space where L* represents a lightness axis, a* represents a red-green axis and b* represents a yellow-blue axis. The L* and b* axes are typically of greatest applicability to tooth whitening, which can be measured as increase in whiteness relative to an untreated surface. Increase in whiteness can be computed from differences in L*, a* and b* values between untreated and treated surfaces. A useful parameter is ΔE*, calculated as the square root of the sum of the squares of differences in L*, a* and b* values, using the formula:
ΔE*=[(ΔL*)²+(Δa*)²+(Δb*)²]^{1/2}
A higher value of ΔE* indicates greater increase in whiteness.

In another embodiment a method of delivering fluoride to a dental surface is provided, the method comprising placing on the surface a film composition of the invention that comprises a source of fluoride ions as described above, wherein during the placing sufficient pressure is applied to promote adhesion of the film to the surface.

In yet another embodiment a method of delivering an antimicrobial agent to a dental surface is provided, the method comprising placing on the surface a film composition of the invention that comprises an antimicrobial agent as described above, wherein during the placing sufficient pressure is applied to promote adhesion of the film to the surface.

In yet another embodiment a method of delivering a desensitizing agent to a dental surface is provided, the method comprising placing on the surface a film composition of the invention that comprises a desensitizing agent as described above, wherein during the placing sufficient pressure is applied to promote adhesion of the film to the surface.

According to any of the above embodiments, the film is left in place until it has substantially dissolved in saliva. The time taken for this dissolution to occur varies depending on the precise composition of the film. If the film dissolves too rapidly, the oral care substance may be inadequately delivered to the dental surface, instead being to a considerable extent removed in saliva from the site of placement of the film. The film should therefore be selected to dissolve in a time period effective to deliver the oral care substance to the dental surface, for example a period of about 5 to about 60 minutes, about 5 to about 30 minutes or about 10 to about 30 minutes as indicated above.

If desired, remnants of the film can later be removed, for example by brushing. In some embodiments, however, the film dissolves so completely that no residue remains for later removal.

Practice of the method can consist of a single application as described herein, or can comprise repeated such applications. In one embodiment the present method is repeated at regular intervals, for example twice or once daily, twice or once weekly, twice or once monthly, in a program or regimen conducted at home and/or in a professional or clinical setting.

The invention can further be understood by reference to the following nonlimiting examples.

### EXAMPLES

### Example 1

Film compositions A-F were prepared having the ingredients shown in Table 1. The compositions were prepared by placing a suitable amount of solvent (ethanol and/or ethyl acetate) in a beaker and adding the polymer components to the solvent with stirring until dissolved or dispersed. The oral care substance (in the present example sodium percarbonate or PVP-H₂O₂) and sodium saccharin were added and stirred for 10 minutes, followed by casting of the resulting mixture at 80° C. for 6-15 minutes to provide a film.

TABLE 1
Film compositions of Example 1

| | Weight % | | | | | |
|---|---|---|---|---|---|---|
| Ingredient | A | B | C | D | E | F |
| ethanol | 2.80 | | 2.02 | 1.55 | 3.50 | 2.00 |
| ethyl acetate | | 2.80 | | | | 2.00 |
| water | | | | | 2.10 | |
| PVP/VA copolymer | 23.52 | 23.52 | 44.41 | 61.97 | 49.05 | 40.00 |
| polyvinylpyrrolidone | 23.52 | 23.52 | | | | |
| polyethylene oxide | | | 10.09 | | 9.81 | |
| PEG 600 | 14.56 | 14.56 | 8.07 | 8.85 | 9.81 | 10.00 |
| rosin (colophony) | | | | | | 5.00 |
| mastic | | | | | | 5.00 |
| shellac | | | | | | 5.00 |
| hydroxyapatite | 10.08 | 10.08 | 10.09 | | | 5.00 |
| PVP-H₂O₂ | | | | 26.54 | | |
| sodium percarbonate | 25.20 | 25.20 | 25.03 | | 25.23 | 25.00 |
| sodium saccharin | 0.16 | 0.16 | 0.30 | 1.00 | 0.50 | 1.00 |

In compositions A to F, the particular PVP/VA copolymer used was Luviskol® VA 37E of BASF, having a VP/VA weight ratio of 30:70. Other PVP/VA copolymers could be substituted if desired.

### Example 2

Effectiveness of film composition A of Example 1 in whitening extracted human teeth was tested. Degree of whitening was measured on the L*a*b* system as established by CIE using a Minolta CR-321 chromameter before and after treatment. Increase in whiteness was determined as ΔE, a higher value of ΔE* indicating a greater increase in whiteness. After 14 successive treatments with the film, ΔE* was 3.4, indicating that the teeth were becoming whiter.

1. A composition for delivery of an oral care substance to a dental surface upon application of the composition thereto, the composition being a flexible film comprising the oral care substance dispersed in a film-forming effective amount of a polymeric matrix having a hydrophilic component and a hydrophobic component in a weight ratio selected such that the film is substantially dissolvable in saliva in a period of time effective for delivery of the oral care substance.

2. The composition of claim 1 wherein said hydrophilic monomers are selected from the group consisting of vinylpyrrolidone, vinyl alcohol, acrylic acid, methacrylic acid and ethylene oxide; and wherein said hydrophobic monomers are selected from the group consisting of methyl methacrylate, ethyl acrylate, dimethylaminoethyl methacrylate, propylene oxide, vinyl caprolactam, vinyl acetate, methacrylamide and vinylimadazole.

3. The composition of claim 1 wherein the polymeric matrix comprises polymerized vinylpyrrolidone and vinyl acetate monomers in a weight ratio of vinylpyrrolidone to vinyl acetate of about 90:10 to about 10:90.

4. The composition of claim 3 wherein said vinylpyrrolidone and vinyl acetate monomers are present in a physical mixture of polyvinylpyrrolidone and polyvinyl acetate homopolymers.

5. The composition of claim 3 wherein said vinylpyrrolidone and vinyl acetate monomers are present in a poly(vinylpyrrolidone/vinyl acetate) copolymer.

6. The composition of claim 5 comprising at least one oral care substance in a total amount of about 0.01% to about 80% by weight and said poly(vinylpyrrolidone/vinyl acetate) copolymer in an amount of about 0.5% to about 70% by weight of the composition.

7. The composition of claim 5 wherein the vinylpyrrolidone to vinyl acetate weight ratio in the copolymer is about 75:25 to about 20:80.

8. The composition of claim 5 wherein the vinylpyrrolidone to vinyl acetate weight ratio in the copolymer is about 60:40 to about 30:70.

9. The composition of claim 5 wherein the poly(vinylpyrrolidone/vinyl acetate) copolymer is present in an amount of about 20% to about 65% by weight of the composition.

10. The composition of claim 5 wherein the polymer matrix further comprises at least one polymer selected from the group consisting of polyvinylpyrrolidone, polyethylene oxide, methylcellulose, ethylcellulose, carbomers and polyacrylates.

11. The composition of claim 5 further comprising at least one natural resin.

12. The composition of claim 5 further comprising a sweetener.

13. The composition of claim 1 wherein the film is substantially dissolvable in saliva in about 5 to about 60 minutes.

14. The composition of claim 1 wherein the film is substantially dissolvable in saliva in about 5 to about 30 minutes.

15. The composition of claim 1 wherein the film is substantially dissolvable in saliva in about 10 to about 30 minutes.

16. The composition of claim 1 wherein the oral care substance is selected from the group consisting of whitening agents, fluoride ion sources, antimicrobial agents, desensitizing agents, anticalculus agents, stannous ion sources, zinc ion sources, antioxidants, sialagogues, breath freshening agents, antiplaque agents, anti-inflammatory agents, periodontal agents, analgesics and nutrients.

17. The composition of claim 1 wherein the oral care substance is a whitening agent selected from the group consisting of peroxy compounds, chlorine dioxide, chlorites and hypochlorites.

18. The composition of claim 1 comprising at least one peroxy compound selected from the group consisting of hydrogen peroxide, peroxides of alkali and alkaline earth metals, organic peroxy compounds, peroxy acids and salts thereof, and polymer-peroxide complexes.

19. The composition of claim 18 wherein the at least one peroxy compound is sodium percarbonate.

20. The composition of claim 1 comprising at least one peroxy compound in a total hydrogen peroxide equivalent amount of about 0.1% to about 50% by weight.

21. The composition of claim 1 wherein the oral care substance is a fluoride ion source selected from the group consisting of fluoride, monofluorophosphate and fluorosilicate salts.

22. The composition of claim 1 wherein the oral care substance is an antimicrobial agent selected from the group consisting of triclosan, 2,2′-dihydroxy-5,5′-dibromodiphenyl ether, 8-hydroxyquinoline and salts thereof, copper (II) compounds, zinc ion sources, phthalic acid and salts thereof, hexetidine, octenidine, sanguinarine, benzalkonium chloride, salicylanilide, domiphen bromide, alkylpyridinium chlorides, iodine, sulfonamides, bisbiguanides, phenolics, piperidino derivatives, magnolia extract, grapeseed extract, phenol, thymol, eugenol, menthol, geraniol, carvacrol, citral, eucalyptol, catechol, 4-allylcatechol, hexyl resorcinol, halogenated bisphenolics, methyl salicylate and antibiotics.

23. The composition of claim 1 wherein the oral care substance is a desensitizing agent selected from the group consisting of potassium citrate, potassium tartrate, potassium chloride, potassium sulfate, potassium nitrate and sodium nitrate.

24. The composition of claim 1 wherein the flexible film comprises a plurality of layers of differing composition.

25. The composition of claim 1 wherein the flexible film comprises a first layer comprising the oral care active dispersed in said polymeric matrix, and a second layer comprising an adhesive.

26. The composition of claim 1, further comprising a backing layer that is non-dissolvable in saliva, said backing layer comprising one or more water-insoluble polymers.

27. A method of delivering an oral care substance to a dental surface, the method comprising placement of the composition of claim 1 on the surface with sufficient pressure to promote adhesion of the composition to the surface.
